(19) United States
(12) Patent Application Publication
(10) Pub. No.: US 2007/0061001 A1
(43) Pub. Date: Mar. 15, 2007

(54) Packaging sheath for drug coated stent

(75) Inventors: Jonathan P. Durcan, Temecula, CA (US); Andrew McNiven, County Tipperary (IE)

Correspondence Address: FULWIDER PATTON LLP, HOWARD HUGHES CENTER, 6060 CENTER DRIVE, TENTH FLOOR, LOS ANGELES, CA, 90045, US

(73) Assignee: Advanced Cardiovascular Systems, Inc.

(21) Appl. No.: 11/225,746

(22) Filed: Sep. 13, 2005

(51) Int. Cl. A61F 2/06

(52) U.S. Cl. 623/1.12

(57) ABSTRACT

A sheath for temporarily protecting the drug coating on the surface of a stent after being mounted on a delivery catheter until just before use. The sheath is configured so as to enable its inner diameter to be temporarily increased to facilitate unrestricted fitment about and removal from the stent. Its inner diameter in its relaxed state is selected to be slightly less that the outer diameter of the mounted stent so as to positively grasp the stent and prevent dislodgement.

### FIELD OF THE INVENTION

The present invention is directed to the protection of a stent prior to its use and more particularly pertains to a device for protecting the surface of a coated stent during its packaging, shipping, subsequent removal from the packaging and handling prior to introduction into a guiding catheter.

### BACKGROUND OF THE INVENTION

Stents are particularly useful in the treatment and repair of blood vessels after a stenosis has been compressed by percutaneous transluminal coronary angioplasty (PTCA), percutaneous transluminal angioplasty (PTA), or removed by atherectomy or other means, to help improve the results of the procedure and reduce the possibility of restenosis. Stents also can be used to provide primary compression to a stenosis in cases in which no initial PTCA or PTA procedure is performed. While stents are most often used in the procedures mentioned above, they also can be implanted on another body lumen such as the carotid arteries, peripheral vessels, urethra, esophagus and bile duct.

In typical PTCA procedures, a guiding catheter or sheath is percutaneously introduced into the cardiovascular system of a patient through the femoral arteries and advanced through the vasculature until the distal end of the guiding catheter is in the aorta. A guidewire and a dilatation catheter having a balloon on the distal end are introduced through the guiding catheter with the guidewire sliding within the dilatation catheter. The guidewire is first advanced out of the guiding catheter into the patient's vasculature and is directed across the arterial lesion. The dilatation catheter is subsequently advanced over the previously advanced guidewire until the dilatation balloon is properly positioned across the arterial lesion. Once in position across the lesion, the expandable balloon is inflated to a predetermined size with a radiopaque liquid at relatively high pressure to displace the atherosclerotic plaque of the lesion against the inside of the artery wall and thereby dilate the lumen of the artery. The balloon is then deflated to a small profile so that the dilatation catheter can be withdrawn from the patient's vasculature and the blood flow resumed through the dilated artery. As should be appreciated by those skilled in the art, while the above-described procedure is typical, it is not the only method used in angioplasty.

In angioplasty procedures of the kind referenced above, abrupt reclosure may occur or restenosis of the artery may develop over time, which may require another angioplasty procedure, a surgical bypass operation, or some other method of repairing or strengthening the area. To reduce the likelihood of the occurrence of abrupt reclosure and to strengthen the area, a physician can implant an intravascular prosthesis for maintaining vascular patency, commonly known as a stent, inside the artery across the lesion. Stents are generally cylindrically shaped devices which function to hold open and sometimes expand a segment of a blood vessel or other arterial lumen, such as coronary artery. Stents are usually delivered in a compressed condition to the target location and then are deployed into an expanded condition to support the vessel and help maintain it in an open position. The stent is usually crimped tightly onto a delivery catheter and transported in its delivery diameter through the patient's vasculature. The stent is expandable upon application of a controlled force, often through the inflation of the balloon portion of the delivery catheter, which expands the compressed stent to a larger diameter to be left in place within the artery at the target location. The stent also may be of the self-expanding type formed from, for example, shape memory metals or superelastic nickel-titanum (NiTi) alloys, which will automatically expand from a compressed state when the stent is advanced out of the distal end of the delivery catheter into the body lumen.

The above described, non-surgical interventional procedures, when successful, avoid the necessity for major surgical operations. However, restenosis of blood vessels, such as coronary vessels treated with PTCA or stents (as described above) has presented a clinical challenge. To address this problem, various coatings have been applied to the stents in order to reduce restenosis by locally delivering drugs to the target site of possible restenosis. The coating may be somewhat frangible which may present a problem in the handling of the stent prior to its use.

In an effort to address such problem, a packaging sheath in form of a short section of tubing has heretofore been fitted to the mounted stent. Such approach has presented a number of problems to the extent that relative movement between the packaging sheath may damage the fragile stent coating. Such damage may occur when slipping the sheath onto the stent or when slipping the sheath off of the stent prior to use. Increasing the sheath's inner diameter is counterproductive as the sheath may then be prone to becoming dislodged during handling or shipping to expose the stent coating to damage and/or cause damage directly as a result of any longitudinal displacement. Additionally, a gap between the sheath and the stent may allow the balloon tapers to expand during the EtO sterilization procedure. Conversely, while decreasing the sheath's inner diameter would prevent inadvertent dislodgement and balloon taper expansion, it would aggravate the potential for damage during fitment and removal of the sheath.

A packaging sheath is needed that minimizes damage to a stent coating during fitment and removal as well precludes inadvertent displacement of the sheath during handling and shipping.

### SUMMARY OF THE INVENTION

The present invention provides a sheath for temporarily protecting a stent after it has been mounted on a delivery catheter and before it is introduced into a patient. Drug coated stents are especially well served by the use of such a sheath as the drug coating may be relatively delicate and easily damaged during handling. The sheath is configured to positively grasp the stent once fitted thereto in order to prevent any dislodgement thereof. This not only prevents the stent or any portion thereof from inadvertently becoming exposed to a damaging contact but also prevents the sheath itself from inflicting damage on the stent coating as a result of relative movement there between. A snug fit also prevents the balloon tapers from expanding during EtO sterilization. Additionally, the sheath is configured to allow it to be easily fitted about and removed from the stent without frictionally engaging the stent surface to thereby further preclude harm from being done to the stent coating by the protective sheath.

The sheath of the present invention consists of a generally cylindrical structure that can be manipulated so as to temporarily increase its inner diameter. The sheath is biased into its reduced diameter configuration wherein such diameter is selected to be slightly less than the outer diameter of the mounted stent it is intended to protect. Such bias is relied upon to enable the sheath to positively grasp the stent once fitted thereto to preclude its dislodgement. By manipulating the sheath so as to overcome its bias towards its reduced diameter configuration, its inner diameter can be increased sufficiently to allow the sheath to fitted to the stent or removed therefrom without any significant contact there between.

In a preferred embodiment of the present invention, two longitudinally hinged cylinder halves are biased into engagement with one another to define an inner diameter that is slightly less than the outer diameter of the mounted stent that the sheath is intended to protect. Two tabs, one extending from each sheath half, allow the two sheath halves to be spread apart by pinching the tabs toward one another. A compression spring extending between the tabs serves to bias the sheath halves into engagement with one another.

In another preferred embodiment of the present invention a cylindrical structure has a helical cut formed therein that extends from the proximal end to the distal end of the sheath. In its relaxed state, the sheath has an inner diameter that is slightly less than the outer diameter of the mounted stent that the sheath is intended to protect. By grasping opposite ends of the sheath and applying a torque, the cut is opened to yield an increase of the inner diameter of the sheath. The natural resilience of the sheath material serves to bias the sheath into its reduced diameter configuration.

These and other features and advantages of the present invention will become apparent from the following detailed description of a preferred embodiment which, taken in conjunction with the accompanying drawings, illustrates by way of example the principles of the invention.

### BRIEF DESCRIPTION OF THE DRAWINGS

FIG. 1 is a cross-sectional view of a protective sheath of the present invention in place about a stent that is mounted on a delivery catheter;

FIG. 2 is a top plan view of a packaged stent and delivery catheter;

FIG. 3 is an enlarged perspective view of a preferred embodiment of the present invention;

FIG. 4 is a cross-sectional view of the embodiment shown in FIG. 3 in place about a mounted stent;

FIG. 5 is a perspective view of the another preferred embodiment of the present invention;

FIG. 6 is a perspective view of the embodiment of FIG. 5 in its enlarged state; and

FIG. 7 is a perspective view of an additional preferred embodiment of the present invention.

### DETAILED DESCRIPTION OF PREFERRED EMBODIMENTS

The present invention provides a device for protecting a stent, and more particularly for protecting the relatively delicate coating thereon, from damage prior to its use. The device is easily fitted to a mounted stent, positively stays in place to protect the stent during its subsequent handling, packaging, shipping, removal from the packaging and preparation for introduction into a guiding catheter. Moreover, the device is configured to eliminate friction between it and the coated surface of a stent during fitment about and removal from the stent yet grasps the stent with sufficient force once fitted to preclude dislodgement.

The protective sheath of the present invention is easily manipulated to temporarily assume an enlarged inner diameter in order to permit it to be longitudinally shifted relative to the stent with only minimal or preferably, without any contact. By substantially reducing or eliminating frictional contact with the stent, the risk of damage to the stent coating is greatly reduced. Once the sheath is allowed to assume its reduced inner diameter, the compressive force exerted thereby serves to substantially preclude any longitudinal shifting to thereby again minimize or preclude damage to the coated surface.

FIG. 1 is an enlarged cross-sectional view of an assembly 12 in accordance with the present invention that includes a delivery catheter 14 having an expandable balloon 16 near its distal end, a stent 18 that is crimped into place about the balloon and a protective sheath 20 in position about the stent. The sheath is shown in its contracted configuration wherein its interior surface fully engages the stent and additionally exerts a compressive force against the stent to prevent longitudinal movement there between.

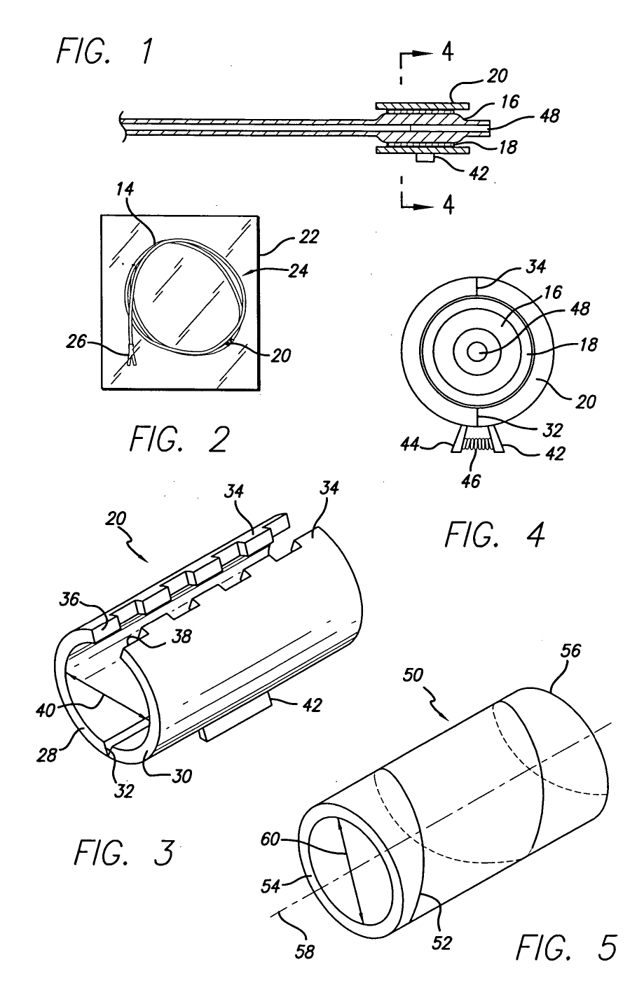

FIG. 2 is a top plan view of a stent and delivery catheter combination sealed within a bubble pack 22. After sterilization, the catheter 14 having a stent mounted thereon and the protective sheath 20 in place is coiled 24 and sealed into a bubble pack. The catheter's proximal end 26 and the sheathed stent 20 is shown in a position. The packaged device is then ready for distribution, can be stored for an extended period of time and just prior to use is removed from the packaging.

FIG. 3 is an enlarged perspective view showing a preferred embodiment of the protective sheath 20 of the present invention. The sheath includes two hinged sheath halves 28, 30 that are biased toward one another. The two halves are joined along a hinge line 32 which may comprise a section of reduced wall thickness and include a set of interlocking teeth 34 that are formed along their opposed edges 36, 38. The device is shown in its open position wherein in its inner diameter 40 has been increased. Radially extending tabs 32, 44 disposed near the hinge line facilitate the manipulation of the device so as to increase its inner diameter.

FIG. 4 is a cross-sectional view taken along lines 4-4 of FIG. 1. The sheath 20, including its two sheath halves 28, 30 is shown in position about a mounted stent 18. The two tabs 42, 44 that are positioned on either side of the hinge line 32 facilitate the enlargement if the inner diameter of the sheath. By pinching the two tabs toward one another and overcoming the bias of spring 46, the interlocking teeth 34 part and the inner diameter of the sheath is increased. Additionally visible is the expandable balloon 16 and a guide wire lumen 48 that extends there through.

After a drug coated stent is crimped onto a collapsed balloon catheter, the sheath 20 shown in FIGS. 3 and 4 is opened by pinching the two tabs 42, 44 toward one another. The opened sheath is then positioned over the stent and released to securely grasp the stent. The delivery catheter including the sheathed stent is then subjected to EtO sterilization, is packaged and distributed to the end user. Just prior to use, catheter is removed from the packaging and the packaging sheath is removed from the stent by again pinching the tabs toward one another to release the stent and then longitudinally displacing the sheath from over the stent. The sheath is discarded and the stent is ready for implantation.

FIG. 5 is a perspective view of another preferred embodiment of the invention in the form of a cylindrical sheath 50 having a helical cut 52 formed therein that extends from its proximal end 54 to its distal end 56. The cut defines an angle of from about 15° to about 45° and preferably about 30° relative to the sheath's axis 58. The sheath is shown in its relaxed state wherein its inner diameter 60 is at its minimum.

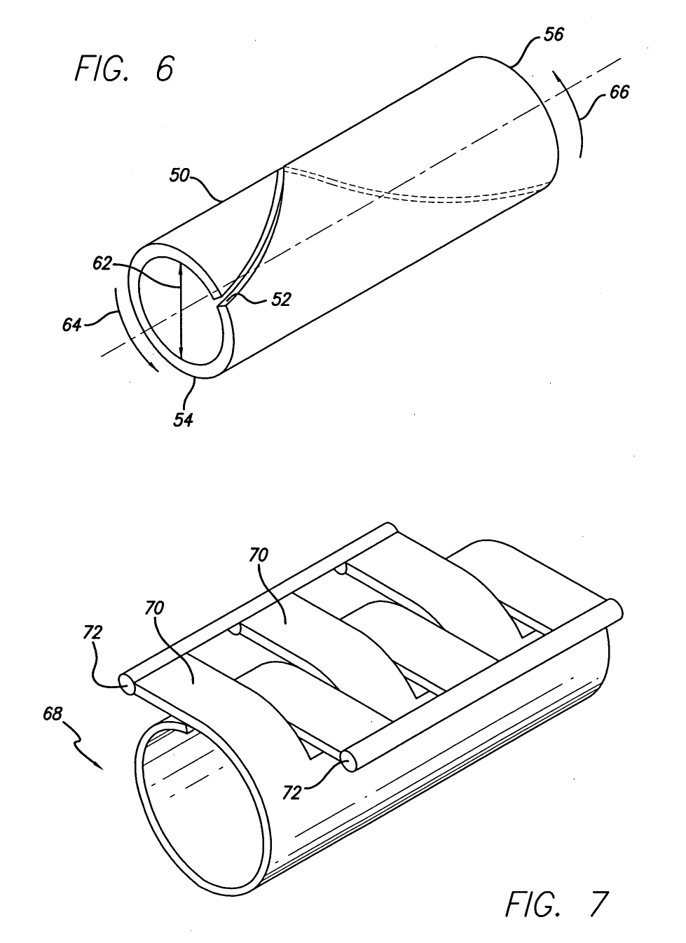

FIG. 6 is a perspective view of the sheath shown in FIG. 5 in its enlarged configuration. With the application of torque to the opposite ends of the sheath, the sheath unwinds slightly so as to effectively increase its inner diameter 62.

FIG. 7 is a perspective view of a further preferred embodiment of the invention in the form of a sheath 68 with intermeshing tabs 70. The tabs are interconnected by a rigid rod 72 so as to enable the inner diameter of the entire length of the sheath to be increased by pinching the rigid rods and thereby urging them towards one another. The resiliency of the material causes the sheath to regain its original reduced inner diameter upon release of the device.

After a drug coated stent is crimped onto a collapsed balloon catheter, the sheath 50 shown in FIGS. 5 and 6 is opened by twisting the ends 54, 56 relative to one another and slipping the sheath into position over the stent. The shallow angle of the cut is advantageous in that the edges of the cut sheath do not tend to catch the edges of the stent during application or removal of the sheath. Upon release, the sheath assumes its relaxed configuration of reduced inner diameter to securely grasp the stent. The angling of the cut line is advantageous in that when the catheter and hence the sheath is bent into the coiled configuration in which it is packaged (FIG. 2) the sheath is able to bend freely and any tendency to open along the cut would be uniformly distributed regardless of the cut's orientation. The delivery catheter including the sheathed stent is then subjected to EtO sterilization, is packaged and distributed to the end user. Just prior to use, catheter is removed from the packaging and the packaging sheath is removed from the stent by again twisting the ends slightly and then longitudinally displacing the sheath from over the stent. The sheath is discarded and the stent is ready for implantation.

A sheath in accordance with the present invention can be configured to accommodate mounted stents ranging from about 1.0 mm to about 10.0 mm in diameter and from about 3.0 mm to about 50 mm in length. The sheath is preferably configured such that its inner diameter is about 0.002″ less than the outer diameter of the mounted stent. The spiral cut sheath may be made of most any injection moldable material and is preferably molded of PTFE, LLDPE, Nylon, Pebax or polyethylene or most preferably, FEP.

While particular forms of the present invention have been illustrated and described, it will also be apparent to those skilled in the art that various modifications can be made without departing from the spirit and scope of the present invention. Accordingly, it is not intended that the invention be limited except by the appended claims.

What is claimed:

1. A sheath for temporarily protecting a mounted stent, comprising:
a generally cylindrical structure having an inner dimension adjustable between a first inner diameter and a second inner diameter, wherein said first inner dimension is slightly less than the outer diameter of said mounted stent and said second dimension is substantially greater than the outer diameter of said mounted stent and wherein said structure is biased toward said first inner diameter.
The sheath of claim 1, wherein said cylindrical structure is longitudinally split along one side and longitudinally hinged along a diametrically opposite side so as to define two opposed sections wherein said opposed sides define said first diameter when in a closed position and said second diameter when in an open position.

2. The sheath of claim 2, wherein each of said opposed sections has a tab extending radially therefrom adjacent said longitudinally hinged side.

3. The sheath of claim 3, wherein a compression spring is disposed between said tabs so as to force said opposed sections against one another.

4. The sheath of claim 2, wherein said opposed sections have interlocking teeth defined along said split side.

5. The sheath of claim 1, wherein said cylindrical structure has helical cut formed there through that extends from its proximal end to its distal end.

6. The sheath of claim 6, wherein said helical cut defines a pitch angle of about 15° to 45°.

7. The sheath of claim 7, wherein said helical split defines a pitch angle of about 30°.

8. The sheath of claim 1, wherein said structure has an inner diameter that is substantially constant along its length.

9. The sheath of claim 1, wherein said structure has an inner diameter that varies along its length.

10. A protected stent assembly, comprising:
a catheter having an expandable balloon;
a drug coated stent crimped onto said balloon;
a protective sheath disposed about said stent, wherein said sheath is manipulatable between a configuration in which its inner diameter is slightly less than said stent's outer diameter and a configuration in which its inner diameter is substantially greater than said stent's outer diameter.

12. The protected stent assembly of claim 11, wherein said sheath comprises a longitudinally hinged cylindrical structure that is biased into said configuration in which its inner diameter is slightly less than said stent's outer diameter.

13. The protected stent assembly of claim 12, wherein said structure is biased by a spring.

14. The protected stent assembly of claim 11, wherein said sheath comprises a cylindrical structure having a helical cut formed there through that extends from its proximal end to its distal end.

15. The protected stent assembly of claim 14, wherein said sheath is constructed of material with an inherent elasticity and said inherent elasticity is relied upon to bias said sheath into said configuration in which its inner diameter is slightly less than said stent's outer diameter.

16. A sheath for temporarily protecting a mounted stent, said mounted stent having an outer diameter, comprising a cylindrical structure defined by two longitudinally hinged cylinder halves each having an opposing edge wherein said cylinder halves define an inner diameter that is slightly less than said stent's outer diameter when said opposing edges engage one another and an inner diameter that is substantially greater than said stent's outer diameter when said opposing edges are forced apart.

17. The sheath of claim 16, wherein said opposing edges are biased toward one another.

18. The sheath of claim 16, wherein each of said opposing edges has teeth formed therein that interlock when said opposing edges engage one another.

19. A sheath for temporarily protecting a mounted stent, said mounted stent having an outer diameter, comprising a cylindrical structure having a helical cut formed there through that extends from a proximal end to a distal end, wherein said cylindrical structure has an inner diameter that is slightly less than said stent's outer diameter when said structure is in its relaxed state and an inner diameter that is substantially greater than said stent's outer diameter when said ends are twisted relative to one another.

20. The sheath of claim 19, wherein said helical cut forms an angle of between about 15° and about 45° relative to said cylindrical structure's longitudinal axis.
